SEQUENCE LISTING (7 sequences)

SEQ ID NO: 1 - protein, 269 aa, Rhizomucor miehei
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

SEQ ID NO: 2 - protein, 538 aa, Rhizopus delemar
SDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCLSDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 3 - protein, 286 aa, Fusarium oxysporum
AVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRRYRSAESVDKR

SEQ ID NO: 4 - protein, 278 aa, Penicillium camemberti
DVSTSELDQFEFWVQYAAASYYEADYTAQVGDKLSCSKGNCPEVEATGATVSYDFSDSTITDTAGYIAVDHTNSAVVLAFRGSYSVRNWVADATFVHTNPGLCDGCLAELGFWSSWKLVRDDIIKELKEVVAQNPNYELVVVGHSLGAAVATLAATDLRGKGYPSAKLYAYASPRVGNAALAKYITAQGNNFRFTHTNDPVPKLPLLSMGYVHVSPEYWITSPNNATVSTSDIKVIDGDVSFDGNTGTGLPLLTDFEAHIWYFVQVDAGKGPGLPFKR

SEQ ID NO: 5 - protein, 269 aa, Thermomyces lanuginosus [sequence omitted]

SEQ ID NO: 6 - protein, 269 aa, Thermomyces ibadanensis
AVPQDLLDQFELFSQYSAAAYCAANNHAPVGSDVTCSENVCPEVDAADATFLYSFEDSGLGDVTGLLALDNTNKLIVLSFRGSRSVENWIANLAADLTEISDICSGCEGHVGFVTSWRSVADTIREQVQNAVNEHPDYRVVFTGHSLGGALATIAAAALRGNGYNIDVFSYGAPRVGNRAFAEFLTAQTGGTLYRITHTNDIVPRLPPRDWGYSHSSPEYWVTSGNDVPVTANDITVVEGIDSTDGNNQGNIPDIPSHLWYFGPISECD

SEQ ID NO: 7 - protein, 261 aa, Aspergillus niger
ASTQGISEDLYNRLVEMATISQAAYADLCNIPSTIIKGEKIYNAQTDINGWILRDDTSKEIITVFRGTGSDTNLQLDTNYTLTPFDTLPQCNDCEVHGGYYIGWISVQDQVESLVKQQASQYPDYALTVTGHSLGASMAALTAAQLSATYDNVRLYTFGEPRSGNQAFASYMNDAFQVSSPETTQYFRVTHSNDGIPNLPPAEQGYAHGGVEYWSVDPYSAQNTFVCTGDEVQCCEAQGGQGVNDAHTTYFGMTSGACTWV

(19) United States
(12) Patent Application Publication
(10) Pub. No.: US 2008/0138856 A1
(43) Pub. Date: Jun. 12, 2008

(54) Enzymatic Enantioselective Ester or Amide Hydrolysis or Synthesis

(75) Inventors: Allan Svendsen, Horsholm (DK); Jesper Vind, Vaerloese (DK); Jesper Brask, Bagsvaerd (DK); Leonardo De Maria, Frederiksberg (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DE)

(21) Appl. No.: 11/813,614

(22) PCT Filed: Feb. 10, 2006
(86) PCT No.: PCT/DK06/00076

§ 371 Date: Jul. 10, 2007

(30) Foreign Application Priority Data
Feb. 10, 2005 (EP) 05388012.6

(51) Int. Cl. C12P 21/06; C12P 7/62

(52) U.S. Cl. 435/68.1; 435/135

(57) ABSTRACT

The enantioselectivity of fungal lipolytic enzymes can be altered by substituting a suitably selected amino acid residue. The residue to be substituted is selected from its location in the 3D structure of the enzyme and an ester substrate (or a substrate analogue). A residue in the lid may be selected if it is located close to the acid part or close to the alcohol part of an ester substrate. A residue outside the lid region may be selected if it is located close to the active site or close to the substrate.

### FIELD OF INVENTION

The present invention relates to an enzymatic method of hydrolyzing or synthesizing a chiral or prochiral carboxylic acid ester or amide. It also relates to variant enzymes and to a method of producing a variant enzyme for use therein.

### BACKGROUND OF THE INVENTION

Enzymatic processes are known to be useful for the enantioselective hydrolysis of chiral or prochiral carboxylic esters, e.g. in the preparation of pharmaceuticals or pesticides. Enzymes used for this purpose include fungal lipolytic enzymes such as lipases from Thermomyces lanuginosus (previously known as Humicola lanuginosa) and Rhizomucor miehei which have a three-dimensional (3D) structure where the active site is covered by a so-called “lid”. M. Holmquist et al, Journal of Protein Chemistry, Vol. 12, No. 6, 1993, pages 749-757 indicates that a substitution of the amino acid residue W89 alters the enantioselectivity.

### SUMMARY OF THE INVENTION

The inventors have found that the enantioselectivity of fungal lipolytic enzymes can be altered by substituting a suitably selected amino acid residue. The residue to be substituted is selected from its location in the 3D structure of the enzyme and an ester substrate (or a substrate analogue). A residue in the lid may be selected if it is located close to the acid part or close to the alcohol part of an ester substrate. A residue outside the lid region may be selected if it is located close to the active site or close to the substrate.

### BRIEF DESCRIPTION OF DRAWINGS

FIG. 1 shows an alignment of amino acid sequences of known fungal lipolytic enzymes SEQ ID NO: 1 to 6, as follows:

1: Rhizomucor miehei (SWISSPROT P19515)

2: Rhizopus delemar (1 tic)

3: Fusarium oxysporum (U.S. Pat. No. 6,103,505 SEQ ID NO: 2, GENESEQP AAW51767, only residues 1-273 shown)

4: Penicillium camemberti (SWISSPROT P25234)

5: Thermomyces lanuginosus (SWISSPROT 059952)

6: Thermomyces ibadanensis (WO2002066622A2)

### DETAILED DESCRIPTION OF THE INVENTION

### Chiral or Prochiral Reactants

The reactants are chiral or prochiral. The reactants for the hydrolysis reaction are the ester or amide and water. Ester- or amide synthesis may occur by reaction of an alcohol or an amine with a carboxylic acid or an activated carboxylic acid. The activated carboxylic acid may be an ester, e.g. vinyl esters.

The ester or amide may be chiral with the general formula R¹—CO—X—R². X is O (oxygen) or NH. R¹ and R² are independently H or hydrocarbyl (optionally substituted), e.g. linear or branched alkyl, aryl or alkaryl, e.g. with 1-20 carbon atoms. R² is not H when X is oxygen. R¹ and/or R² is chiral (contains a chiral carbon atom). Substituents may be OH; alkoxy residues with particularly 1 to 10 C atoms, particularly methoxy and ethoxy; aryloxy residues with particularly 6 to 14 C atoms, in particular phenoxy; or halogen, particularly fluorine, chlorine or bromine.

R¹ may be R³R⁴R⁵C—. R² may be a primary alkyl of the formula —CH₂—CR⁶R⁷R⁸, or it may be a secondary alkyl of formula —CHR⁹R¹⁰. R³, R⁴, R⁵ R⁶, R⁷, R⁸, R⁹ and R¹ are independently selected among H and hydrocarbyl as defined above. R³, R⁴ and R⁵ may be different, thus making R¹ chiral. R⁶, R⁷ and R⁸ may be different, or R⁹ and R¹⁰ may be different hydrocarbyl (optionally substituted), making R² chiral.

Some examples of chiral acyl R¹—CO are ibuprofen (2-(4-isobutylphenyl)propionic acid), 2-isobutylsuccinic acid, 2-methyl fatty acids with 4-20 carbon atoms, e.g. 2-methyl-butyric acid or 2-methyldecanoic acid. The compound may be an ester (X═O), and the alkyl R² may be methyl, ethyl, 1-hexyl, 1-heptyl, phenyl or p-nitrophenyl.

Some examples of chiral alkyl R² are secondary alcohols such as 2-butanol, 2-hexanol, 3-hexanol or 1-phenyl-ethanol. The compound may be an ester (X═O), and the acyl R¹—CO may be acetate or propionate.

Some particular amides are amino acid amides, eg dipeptides or N-acetyl amino acids.

The ester or amide may be a prochiral meso-form derived from a diacid, a diol or a diamine. Thus, the reaction may be enantioselective hydrolysis of a meso-diester or meso-diamide, or it may be enantioselective synthesis from a (optionally activated) meso-diacid, a meso-diol, or a meso-diamine.

### Parent Polypeptide

The variants used in the invention may be derived from a parent polypeptide which has a high degree of homology to Thermomyces lanuginosus lipase (SEQ ID NO: 5) and/or Rhizomucor miehei lipase (SEQ ID NO: 1). The degree of homology may be at least 50%, at least 60%, at least 70%, at least 80%, at least 90% or at least 95%. The parent polypeptide may be a fungal lipolytic enzyme, and may particularly have a homology of at least 80% (or 85, 90 or 95%) to any of SEQ ID NO. 1-7. SEQ ID NO: 1-6 are identified above. SEQ ID NO: 7 is the feruloyl esterase from Aspergillus niger.

The parent polypeptide has hydrolase activity on a carboxylic ester or amide, and is typically a fungal lipolytic enzyme. It includes an active site, typically a catalytic triad.

### Three-Dimensional Structure of Parent Polypeptide

The invention relies on a three-dimensional (3D) structure of the parent polypeptide together with a substrate or a substrate analog. Examples of known 3D structures (available in the PDB Protein Data Bank at http://www.rcsb.org/pdb/) include the following:

| Structure | Polypeptide | Substrate or analog |
|---|---|---|
| 1GT6 | S146A variant of Thermomyces | Oleic acid (OLA) |
| | lanuginosus lipase | |
| | (SEQ ID NO: 5) | |
| 5TGL | Rhizomucor miehei lipase | N-Hexylphosphonate |
| | (SEQ ID NO: 1) | Ethyl Ester |
| 1UZA, 1UWC, | Aspergillus niger feruloyl | N-Acetylglucosamine |
| 1USW | esterase (SEQ ID NO: 7) | |

The 3D structure generally includes an active site, particularly a catalytic triad, e.g. corresponding to S146, D201 and H258 of SEQ ID NO: 5 in 1GT6. The structure also generally includes a so-called lid, i.e. a movable part which in a closed state covers the active site, e.g. corresponding to amino acid residues 81-100 of SEQ ID NO: 5 in 1GT6.

### Residue Sets

The procedure for selecting amino acid residues for substitution is described below with reference to the B chain in the three-dimensional PDB structure 1GT6 (available at http://www.rcsb.org/pdb/) which includes the S146A variant of Thermomyces lanuginosus lipase (SEQ ID NO: 5) and oleic acid (OLA) as a substrate analogue.

### The Alcohol and Acid Parts of the Lid

The lid is defined to span the residues comprised between 82 and 97. The procedure explained below gives an alcohol part consisting of residues 82, 83, 84, 85 and 88 and an acid part consisting of residues 90, 91, 92, 93, 94, 95, 96 and 97.

### Set of Residues

It is built in the following way. Residues in the lid will not be considered, i.e. residues from 82 to 97 are left out from the following sets. Residues having any heavy atom (i.e. an atom other than H) located within 10 Å from a heavy atom of residue A146, D201 and H258 are grouped together with residues located within 10 Å from the OLA molecule. (Chain B is taken from 1 GT6). The so obtained set is completed adding the residues in the alcohol and the acid part of the lid, i.e. 82, 83-85, 88, 90-97. This procedure selects the following residues: 13, 14, 17, 18, 20, 21, 79-85, 88, 90-97, 109-113, 143-154, 168-177, 195-208, 211, 213, 215, 219-227, 246-249, 251-268.

A procedure based on two planes (described below in the section “Derivation of the equation of the planes”) has been devised to unambiguously assign the residues to one of two space regions called the “alcohol part” and the “acid part” of the space. Residues in the interface of the two regions will be assigned to both. The residues selected above are assigned as follows:

Acid Part of Residue Set

13, 80, 90-97, 109-113, 143-144, 146-154, 168-177, 195-208, 211, 213, 215, 219-223, 246-249, 251-253, 261.

Alcohol Part of Residue Set

13, 14, 17, 18, 20, 21, 79, 80-85, 88, 143-146, 148, 168-172, 196-201, 220-227, 254-268.

### Sub-Set of Residues

It is built in the following way. Residues in the lid will not be considered, i.e. residues from 82 to 97 are left out from the following sets. Residues having any heavy atom located within 6 Å from the OLA molecule. (Chain B is taken from 1GT6). The so obtained set is completed adding the residues in the alcohol and the acid part of the lid, i.e. 82, 83-85, 88, 90-97. This procedure select the following residues: 21, 82-85, 88, 90-97, 110, 113,145-148, 172, 174, 202, 203, 206, 207, 255, 258, 259, 265, 266. The subset is assigned to the acid part and the alcohol part as follows:

Acid Part of the Second Subset

90-97, 110, 113,146-148, 172, 174, 202, 203, 206, 207.

Alcohol Part of the Second Subset

21, 82-85, 88, 145, 146, 148, 172, 255, 258, 259, 265, 266.

### Derivation of the Equation of the Planes

Planes 1 and 2 are described below by reference to the Cartesian coordinates of Chain B in the PDB structure 1GT6.

Plane 1

This is the plane defined by the atoms CA from A146 (atom 1), CG from D201 (atom 2) and C6 from the oleic acid molecule (OLA) (atom 3). We have then:
r₁=(47.800, 15.573, 30.012)
r₂=(41.417, 10.978, 28.949)
r₃=(49.353, 11.751, 32.442)

The equation for the plane gives in this case:

0.404x−0.368y−0.837z+11.531=0.000

Acid Part

A residue is said to belong to the acid part if it has at least one heavy atom at position r=(x,y,z), with its cartesian coordinates satisfaying the following inequality:

0.404x−0.368y−0.837z+11.531≦0.000

Plane 2

This is the plane defined by the atoms CA from A146 (atom 1), CB from S83 (atom 2) and C8 from the oleic acid molecule (OLA) (atom 3). We have then:
r1=(47.800, 15.573, 30.012)
r2=(49.710, 11.872, 29.963)
r3=(53.679, 11.269, 30.115)

The equation for the plane gives in this case:

0.044x+0.036y−0.998z+27.320=0.000

Alcohol Part

A residue is said to belong to the alcohol part if it has at least one heavy atom at position r=(x,y,z), with its cartesian coordinates satisfaying the following inequality:

0.044x+0.036y−0.998z+27.320>0.000

### Substitution of Selected Residue

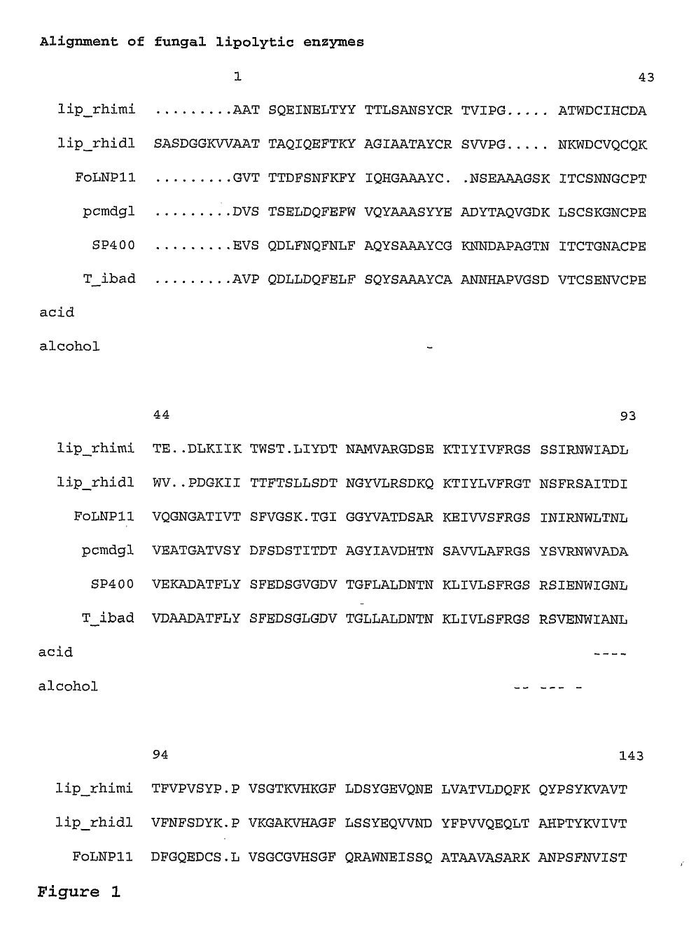

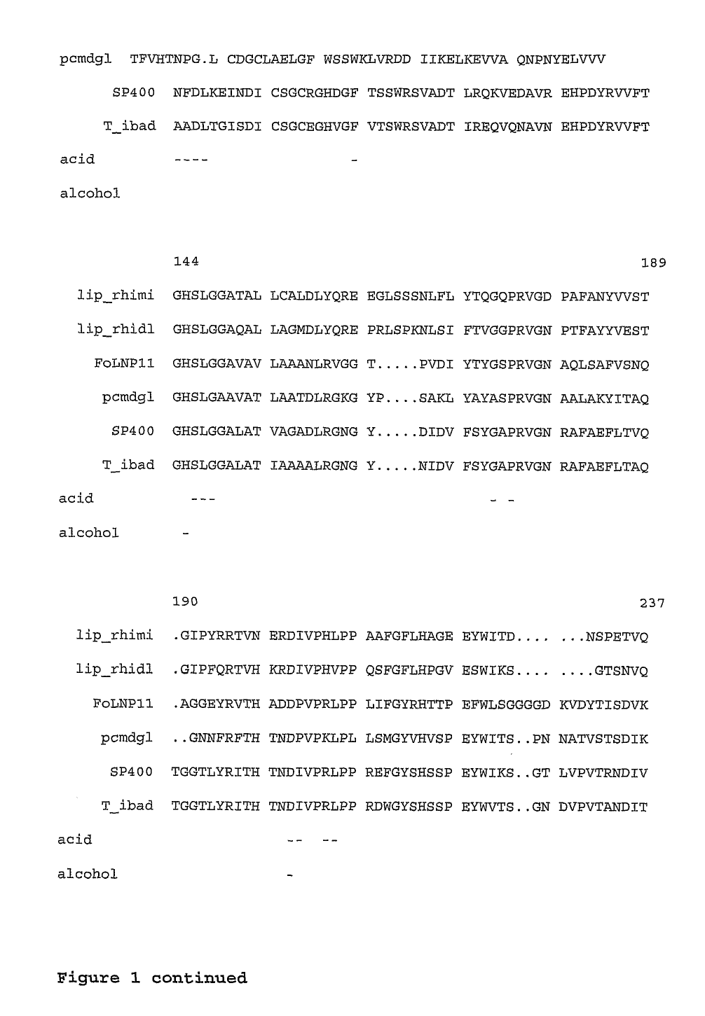

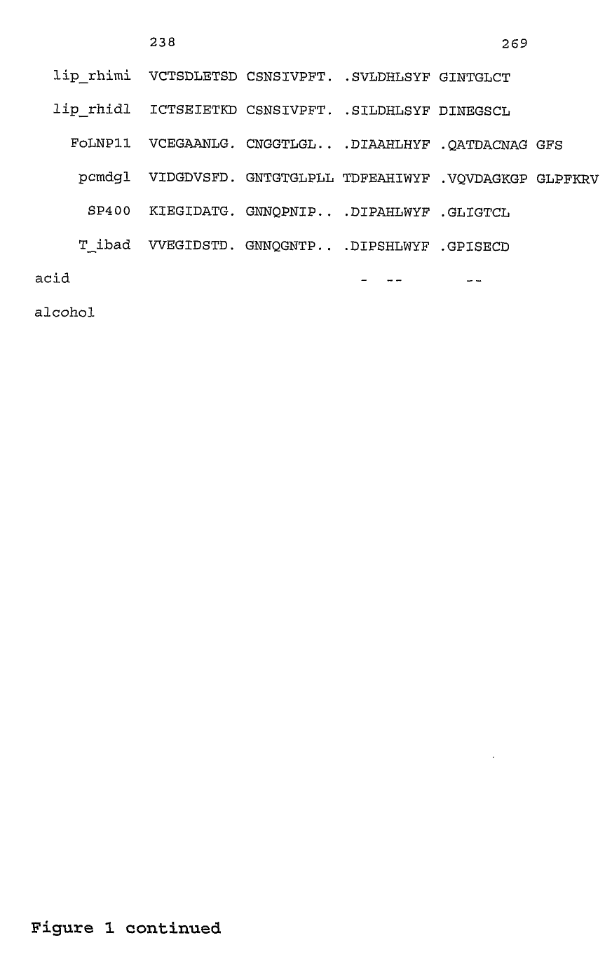

The amino acid residue to be substituted may be selected in SEQ ID NO: 5 as described above, or a corresponding residue in another parent polypeptide may be selected based on an alignment with SEQ ID NO: 5. FIG. 1 shows an alignment of SEQ ID NO: 1-6. Other sequences may be aligned as described below.

The selected residue may be substituted with a smaller residue or a residue of nearly identical size, in order to better accommodate the substrate. Amino acid residues are ranked as follows from smallest to largest: (an equal sign indicates residues with nearly identical sizes):

G<A<S=C<V=T<P<L=1=N=D=M<E=Q<K<H<R<F<y<W

The variant may comprise one or more substitutions corresponding to the following in SEQ ID NO: 5 (Thermomyces lanuginosus lipase): S83T, R84GRWK, I90Q, G91IAN, N92TD, L93T, N94R, F95LY, D96W, F113Y, P174C, 1202M, V203SAGTM, L206T, F211E, L227G, 1255N, P256T, L259T, G263Q, L264A, 1265T, G266DW, T267A.

The variant may comprise one or more substitutions corresponding to the following in SEQ ID NO: 3 (lipase/phospholipase from Fusarium oxysporum): I83NGLSY, D265AGYE, The notation used here is that S83T indicates a substitution of S (Ser) at position 83 with T (Thr). R84GRWK indicates a substitution of R84 with any one of residues G, R, W or K.

### Optional Amino Acid Modifications

In addition to substitution of one more selected residues, the variant may further comprise one or more substitutions corresponding to the following in SEQ ID NO: 5: D27R, D111A, S216P, E87T, W89L, T231R, N233R,

### Particular Variants

Variants may be derived from SEQ ID NO: 5 by making one of the following sets of substitutions:

I202M, T231R, N233R
V203M, T231R, N233R
R84G, F113Y, F211E, I255N
G91A, L93T, F95L, D96W, E99K, V203S, L206T, G263Q,
L264A, I265T, G266D, T267A, L269N
D27R, G91N, N94R, D111A, P174C, S216P, L227G, P256T
F211E, F95Y
F95Y, F211E, I255N
S83T, R84G, I255N
V60G, D62A, S83T, D96W, G266D
V60G, D62A, S83T, D96W, G266W
I86D, W89L, I90Q, L93T
S83T, R84G, I86D, E87T, W89L, I90Q, G91I, N92D, L93T,
F95Y, F113Y, F211E

Variants may be also derived from SEQ ID NO: 3 by making one of the following sets of substitutions:

A29P, I83N
K33N, D265A
K33N, I83G, D265G
K33N, I83L, D265Y
K33N, I83S
K33N, I83Y, D265E

### Altered Enantioselectivity

The altered enantioselectivity may be an increased enantioselectivity for the R or S form. Enantioselectivity may be measured as enantiomeric excess, ee=% enantiomer-A−% is enantiomer-B. To compare the performance of different enzymes, parallel reactions may be performed and the enantiomeric excess measured after a fixed amount of time or after a certain conversion is reached, e.g. 40% product formation.

### Hydrolysis or Synthesis Reaction

Ester or amide hydrolysis may be performed in aqueous buffer, or in mixtures of water and water-miscible organic solvents. In the case of a water-insoluble ester or amide, the hydrolysis process may be performed in a two-phase system consisting of an aqueous phase and a non-miscible organic phase with stirring. A non-ionic surfactant such as an alcohol ethoxylate (e.g. Triton X-100) or an alcohol (such as MeOH, EtOH and/or i-PrOH) may be added to ensure that the polypeptide has the lid in an open configuration.

Ester- or amide synthesis may be performed by adding the enzyme to the reactants as a solution or in dry form, or by use of immobilized enzyme, e.g. on resin beads. The synthesis reaction is generally performed at low water content in the absence or presence of an organic solvent such as a hydrocarbon. In the non-aqueous medium, the polypeptide will generally have the lid in an open configuration.

### Amino Acid Identity, Homology and Alignment

The amino acid identity may be suitably determined by means of computer programs known in the art, such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45), using GAP with the following settings for polypeptide sequence comparison: GAP creation penalty of 3.0 and GAP extension penalty of 0.1.

The variant polypeptide has an amino acid identity to SEQ ID NO: 1 or 5 which is at least 50%, particularly at least 55%, at least 60%, at least 65%, at least 70%, at least 75%, at least 80%, at least 85%, at least 90%, at least 95%, or at least 98%.

To find the homologous positions in lipase sequences not shown in the alignment, the sequence of interest is aligned to the sequences shown in FIG. 1. The new sequence is aligned to the present alignment in FIG. 1 by using the GAP alignment to the most homologous sequence found by the GAP program. GAP is provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45). The following settings are used for polypeptide sequence comparison: GAP creation penalty of 3.0 and GAP extension penalty of 0.1.

Alternative computer programs include the following:

ALIGN0, described at http://evol.mcmaster.ca/Pise/5.a/align0.html Pearson, W. R. (1999) Flexible sequence similarity searching with the FASTA3 program package. Methods in Molecular Biology

W. R. Pearson and D. J. Lipman (1988), Improved Tools for Biological Sequence Analysis, PNAS 85:2444-2448

W. R. Pearson (1998) Empirical statistical estimates for sequence similarity searches. In J. Mol. Biol. 276:71-84

Pearson, W. R. (1996) Effective protein sequence comparison. In Meth. Enz., R. F. Doolittle, ed. (San Diego: Academic Press) 266:227-258

### EXAMPLES

### Example 1

### Synthesis of 2-butyl propionate

The enantioselectivity was tested for variants of T. lanuginosus lipase (SEQ ID NO: 5) and F. oxysporum lipase/phospholipase (SEQ ID NO: 3). The parent enzymes were also tested for comparison.

Immobilized enzymes were used to catalyze the transesterification of vinyl propionate with the secondary alcohol 2-butanol in hexane. Results in terms of conversion and enantiomeric excess (ee) were analyzed by chiral gas chromatography (GC), similar to the method described by S. Patkar et al. in Chem. Phys. Lipids 1998, 93, 95-101.

### Immobilization

More specifically, purified enzymes were immobilized on Accurel polypropylene in a concentration of 20 mg/g. Accurel was initially wetted with EtOH, then filtered and washed with purified water (MQ-water). Lipase solution was then added, as well as 0.1 M phosphate buffer, pH 7, to give a final volume of 10 mL/250 mg Accurel. After gentle shaking for 18 h at room temperature, the preparations were filtered, washed with MQ-water, and dried in vacuum for 48 h. No residual lipase activity was found in the filtrate, indicating a quantitative immobilization.

### Reactions

To immobilized enzyme (10 mg) was added 2-butanol (183 micro-L, 2 mmol), vinyl propionate (220 micro-L, 2 mmol), and hexane (600 micro-L).

Reactions were incubated at 40° C., 1000 rpm. Samples were withdrawn after 1 h or 22 h for analysis by chiral GC (5 micro-L reaction mixture in 1 mL diethyl ether). For some reactions, samples were also withdrawn for NMR analysis (25 micro-L reaction mixture in 0.7 mL CDCl₃).

### Analysis

NMR was performed on a Varian MercuryVX 400 MHz system. GC was performed on a Varian Chrompack CP-3800 fitted with a Varian CP-Chiralsil-DEX CB 10 m column. A temperature program running from 40° C. over 70° C. (2° C./min) to 200° C. (10° C./min) gave good separation of ester enantiomers. Conversions were calculated from NMR results, by comparing integrals of the Hα (CHOH) proton of the alcohol reactant with —COCH₂— integrals from the ester product. The same conversions could be calculated from the GC chromatograms, applying a detector response factor 0.6 for ester products. Hence, with A1 and A2 indicating integrals for the two alcohol enantiomers, and E1 and E2 indicating integrals of the two ester enantiomers, calculations were performed as:

Conv=100%*0.6*(E1+E2)/(0.6*(E1+E2)+A1+A2)

ee=100%*(E1−E2)/(E1+E2)

### Results

The results are given as conversions and ee (enantiomeric excess) for the lipase catalyzed reactions. Selectivity is (R) unless otherwise specified.

| | | Reaction | Conv. | |
|---|---|---|---|---|
| Parent lipase | Substitutions | time (h) | (%) | ee (%) |
| T. lanuginosus | | 22 | 23 | 7 (S) |
| T. lanuginosus | F211E, F95Y | 22 | 18 | 11 (S) |
| T. lanuginosus | F95Y, F211E, I255N | 22 | 31 | 10 (S) |
| T. lanuginosus | I202M, T231R, N233R | 22 | 27 | 8 (S) |
| T. lanuginosus | V203M, T231R, N233R | 22 | 10 | 14 (S) |
| T. lanuginosus | S83T, R84G, I255N | 22 | 3 | 9 |
| F. oxysporum | | 1 | 22 | 3 (S) |
| F. oxysporum | | 22 | 91 | |
| F. oxysporum | A29P, I83N | 22 | 29 | 7 |
| F. oxysporum | K33N, I83L, D265Y | 22 | 28 | 13 |
| F. oxysporum | K33N, I83Y, D265E | 22 | 14 | 9 |

The results indicate that for variants with substitutions only in the alcohol part, the selectivity was inverted (from S to R). For variants with substitutions only or mainly in the acid part; the S-selectivity was retained and increased.

### Example 2

### Ester Hydrolysis

An aqueous solution of lipase (0.1-10 mg) is added to a vigorously stirred suspension of 1-phenyl-ethanol propionic acid ester (1 mmol) in a 10 mM phosphate pH 7 buffer (10 mL) containing 0.4% Triton X-100. Throughout the reaction, pH is maintained at 7 by automatic addition of 1 M NaOH (pH-stat setup). After addition of 0.4 mmol NaOH (corresponding to 40% conversion), the reaction mixture is extracted with CH₂Cl₂ (10 mL). The organic phase is dried (Na₂SO₄) and concentrated to dryness. A sample of the residue (2 micro-L) is dissolved in Et₂O (1 mL) and analyzed by chiral GC as described in Example 1.

1-16. (canceled)

17. A method of hydrolyzing or synthesizing a carboxylic acid ester or amide from chiral or prochiral reactants, comprising:
(a) contacting reactants for the hydrolysis or synthesis with a polypeptide which
(i) has hydrolytic activity on the ester or amide
(ii) has an amino acid sequence which is at least 50% homologous to SEQ ID NO. 5, and
(iii) compared to SEQ ID NO: 5 comprises a different amino acid residue at a position corresponding to any of 13, 14, 17, 18, 20, 21, 79-85, 88, 90-97, 109-113, 143-154, 168-177, 195-208, 211, 213, 215, 219-227, 246-249, or 251-268.

18. The method of claim 17, wherein the polypeptide is at least 80% homologous to any of SEQ ID NOs: 1-6.

19. The method of claim 17, wherein the polypeptide comprises a different amino acid at a position corresponding to any of 13, 80, 90-97, 109-113, 143-144, 146-154, 168-177, 195-208, 211, 213, 215, 219-223, 246-249, 251-253, or 261.

20. The method of claim 17, wherein the polypeptide comprises a different amino acid at a position corresponding to any of 13, 14, 17, 18, 20, 21, 79, 80-85, 88, 143-146, 148, 168-172, 196-201, 220-227, or 254-268.

21. The method of claim 17, wherein the polypeptide comprises a different amino acid at a position corresponding to any of 21, 82-85, 88, 90-97, 110, 113, 145-148, 172, 174, 202, 203, 206, 207, 255, 258, 259, 265, or 266.

22. The method of claim 17, wherein the polypeptide comprises a different amino acid at a position corresponding to any of 90-97, 110, 113, 146-148, 172, 174, 202, 203, 206, or 207.

23. The method of claim 17, wherein the polypeptide comprises a different amino acid at a position corresponding to any of 91, 94-95, 174, 202-203, and 206-207.

24. The method of claim 17, wherein the polypeptide comprises a different amino acid at a position corresponding to any of 21, 82-85, 88, 145, 146, 148, 172, 255, 258, 259, 265, 266.

25. The method of claim 17, wherein the polypeptide comprises a different amino acid at a position corresponding to any of 21, 82-85, 255, 259, or 265-267.

26. The method of claim 17, wherein the ester has an alcohol part having a chiral or prochiral carbon atom.

27. The method of claim 17, wherein the ester has an acid part having a chiral or prochiral carbon atom.

28. The method of claim 17, wherein the selected residue is located on the acid side of a plane defined by atoms corresponding to S146 CA atom, D201 CG atom and OLA C6 atom.

29. The method of claim 17, wherein the selected residue is located on the alcohol side of a plane defined by atoms corresponding to S146 CA atom, S83 CS atom and OLA CS atom.

30. A method of hydrolyzing or synthesizing a carboxylic acid ester or amide from chiral or prochiral reactants, comprising:
(a) providing a parent polypeptide which:
(i) has hydrolase activity on an ester or amide substrate, and
(ii) has an amino acid sequence which is at least 50% homologous to SEQ ID NO., 5 and comprises a catalytic triad corresponding to S146, D201 and H258 and a residue corresponding to S83 of SEC ID NO: 5,
(b) providing a three-dimensional structure corresponding to 1GT6 of the polypeptide and a substrate or a substrate analogue corresponding to OLA in 1GT6 which structure comprises a lid region corresponding to residues 82-97 of SEQ ID NO: 5,
(c) preparing a variant polypeptide having an amino acid sequence which comprises a substitution of an amino acid residue in the polypeptide which in the 3D structure:
(i) is not located in the lid region and has a non-hydrogen atom located within 10 Å of a non-hydrogen atom of the substrate or substrate analogue or of the catalytic triad, or
(ii) is located in the lid region on the acid side of a plane defined by atoms corresponding to S146 CA atom D201 CG atom and OLA C6 atom, or
(iii) is located in the lid region on the alcohol side of a plane defined by atoms corresponding to S146 CA atom, S83 CS atom and OLA CS atom.
(d) contacting the variant polypeptide with the reactants, and
(e) selecting a polypeptide which has an enantioselectivity which is different from the parent polypeptide.

31. A polypeptide which has hydrolase activity on an ester or amide substrate, and has an amino acid sequence which has at least 80% identity to SEQ ID NO: 5 and compared to SEQ ID NO: 5 comprises a substitution corresponding to 190Q, G91I, N92TD, F95Y, F113Y, I202M, V203GM, L269T, 270F.

32. The polypeptide of claim 31, which comprises one of the following sets of substitutions:
I202M, T231R, N233R
V203M, T231R, N233R
R84G, F113Y, F211E, I255N
F211E, F95Y
F95Y, F211E, I255N
I86D, W89L, I90Q, L93T
S83T, R84G, I86D, E87T, W89L, I90Q, G91I, N92D, L93T,
F95Y, F113Y, F211E
